(19) United States
(12) Patent Application Publication
(10) Pub. No.: US 2019/0076829 A1
(43) Pub. Date: Mar. 14, 2019

(54) METHOD FOR THE HYDROTHERMAL PREPARATION OF MOLYBDENUM-BISMUTH-COBALT-IRON-BASED MIXED OXIDE CATALYSTS

(71) Applicant: Evonik Degussa GmbH, Essen (DE)

(72) Inventors: Paul SPRENGER, Karlsruhe (DE); Jan-Dierk GRUNWALDT, Stutensee (DE); Wolfgang KLEIST, Bochum (DE); Achim FISCHER, Goldbach (DE)

(73) Assignee: Evonik Degussa GmbH, Essen (DE)

(21) Appl. No.: 16/084,843

(22) PCT Filed: Mar. 13, 2017
(86) PCT No.: PCT/EP2017/055814

§ 371 Date: Sep. 13, 2018

(30) Foreign Application Priority Data
Mar. 14, 2016 (EP) 16160200.8

(51) Int. Cl. B01J 23/887; B01J 23/89; B01J 35/00; B01J 35/10; B01J 37/08; B01J 37/06; C07C 45/35

(52) U.S. Cl. CPC B01J 23/8876; B01J 23/8993; B01J 35/0006; B01J 35/006; B01J 35/1004; B01J 2523/845; B01J 37/06; C07C 45/35; B01J 2523/54; B01J 2523/68; B01J 2523/842; B01J 37/08

(57) ABSTRACT

The present invention relates to a process for preparing molybdenum-bismuth-iron-cobalt-based multielement oxide catalysts by means of hydrothermal synthesis, wherein the hydrothermal synthesis is conducted with an aqueous solution and/or an aqueous suspension of precursor compounds of the elements present in the multielement oxide catalyst to be prepared, the pH of which has been adjusted to a value between about 6 and about 8. The present invention also further relates to the multielement oxide catalysts obtainable by this process and to the use thereof in the partial gas phase oxidation of olefins and tert-butanol.

The present invention relates to a process for preparing multielement oxide catalysts by means of hydrothermal synthesis, to the multielement oxide catalysts obtained or obtainable by this process, and to the use thereof in the partial gas phase oxidation of olefins.

Multielement oxide catalysts, especially those based on molybdenum oxides and bismuth oxides, are used in the industrial preparation of acrolein and acrylic acid or methacrolein and methacrylic acid by partial oxidation of, respectively, propene and isobutene (2-methyl-1-propene) or tert-butanol (2-methyl-2-propanol), and also of acrylonitrile by ammoxidation of propene. Since the development of bismuth molybdate catalysts for these reactions by the Standard Oil of Ohio company (Sohio for short) in 1959, these mixed oxide or multielement oxide catalysts have gained great attention, and their catalytic properties have therefore been studied in detail. These studies relate to the influence of further elements in addition to bismuth and molybdenum on yield and selectivity for the formation of acrolein or acrylonitrile, and the stability of the catalyst in question in the reaction. One result of these studies was that, irrespective of further elements, the surface layer of these multielement oxide catalysts is always based on oxides of bismuth and molybdenum. Therefore, these elements are considered to be the active elements of these catalysts.

Typically, multielement oxide catalysts are prepared by means of what is called coprecipitation, where precursor compounds of the elements of the catalyst to be prepared are dissolved in water, then a catalytic active composition comprising these elements is precipitated out of this solution and this active composition is then dried and calcined. In addition, multielement oxide catalysts, albeit to a much lesser degree, are also prepared by solid-state syntheses, sol-gel syntheses and spray-drying from solutions comprising precursor compounds of the multielement oxide catalyst to be prepared. The multielement oxide catalyst obtained from these syntheses should have a maximum surface area in order to be able to assure high activity in the catalysed reaction. In general, a high activity is achieved in multielement oxide catalysts by virtue of the catalysts being in the form of crystalline materials. For provision of crystalline materials by means of the aforementioned syntheses, however, high temperatures, especially temperatures of more than 400° C., are always required, whether to be able to convert the starting compounds to the desired multielement oxides at all, for example the conversion of nitrates to the corresponding oxides, or for the final calcination of the catalyst. However, such high temperatures cause a deterioration in the catalytic performance of the resulting phase of the mixed oxide. It is believed that this effect is attributable to enrichment of bismuth in the catalyst surface.

The U.S. Pat. No. 4,418,007 A discloses the preparation of mixed oxide catalysts comprising the oxides of molybdenum and/or tungsten, where in the first stage an aqueous suspension with salts of all metals to comprise the active catalyst is prepared, and the pH of this solution is adjusted to a value of from 6 to 8 by adding ammonia, followed by filtering the suspension to obtain a paste of the desired active catalyst, and in the second stage said paste is dried and at least one calcined.

The U.S. Pat. No. 4,166,808 A discloses a process for the preparation of a catalyst with an active phase corresponding to the formula Mo_{12 2}Co₁₀Fe₁B₁Oₓ, where a solution of precursor compounds of the metals molybdenum, cobalt, iron, and bismuth is provided in the first step, said solution has a pH of 1.1. Said solution is heated to evaporate to 80° C. to evaporate the water to obtain a non-liquid paste, which is further heated to obtain a solid which is subsequently calcined and further processed to give a supported catalyst.

The U.S. Pat. No. 5,245,083 A discloses the preparation of a mixed oxide catalyst, which is obtained by the combination of two compositions, which are prepared by co-precipitation from aqueous solutions of each different metal salts, followed by calcination. The mixed oxide catalyst is obtained by mixing the two compositions in a specific ratio and with an amount of water so that a suspension is obtained, which is then evaporated to dryness and calcined to give the final catalyst.

Also the published US patent application 2009030230 A discloses the preparation of a mixed oxide catalyst by coprecipitation of solids from aqueous solutions containing salts of all the metals which are also to be in the final catalyst.

A further problem with the standard multielement oxide catalysts prepared by means of coprecipitation is their time-limited period of use. The catalysts age within this time-limited period of use, meaning that they no longer have the same catalytic activity at a particular time as at the start of their use in the catalysed reaction. In this case, the aged or spent fixed catalyst bed has to be exchanged for fresh catalyst.

The ageing of a mixed oxide or multielement oxide catalyst having an active composition containing molybdenum in the oxidized state is observed especially when this catalyst is being used in a partial gas phase oxidation in which steam is present or forms. In most cases, the steam is formed in the partial gas phase oxidation. It is believed that the steam present or formed in the partial gas phase oxidation forms MoO₂(OH)₂, which is volatile, with the molybdenum present in oxidized form in the active composition of the catalyst. The specialist literature therefore takes the view that, during the long-term operation of a heterogeneously catalysed gas phase oxidation, molybdenum sublimes continuously out of the active mass of the multielement oxide catalyst as MoO₂(OH)₂ (cf. Investigations of the mechanism and kinetics leading to a loss of molybdenum from bismuth molybdate catalysts, L. Zhang et al., Catalysis A: General (1994), 117, 163-171). The loss of the active molybdenum component in mixed oxide catalysts is at its greatest especially in the region of what are called the hotspots of the fixed catalyst bed, i.e. the points where the evolution of heat of reaction that proceeds over the fixed catalyst bed in flow direction of the reaction gas mixture is at its greatest. A fixed catalyst bed may have a plurality of hotspots. After passing through these regions, the temperature of the fixed catalyst bed and its environment in flow direction of the gases also falls significantly, such that molybdenum oxide is precipitated again as a thin coating or film at the colder points on the inner wall of the reaction tube (cf. EP 2 155 376 A1). Once a region of the fixed catalyst bed has been subject to bleeding of molybdenum from the active component, a hotspot migrates along the fixed catalyst bed further into regions where the temperature of the fixed catalyst bed in flow direction of the gases was previously lower and which therefore have not yet been subject to bleeding of molybdenum. These regions or zones also gradually become deficient in molybdenum. Because of the zone-by-zone deficiency/bleeding of the active molybdenum component in the catalyst, this process is also referred to as zone ageing (band ageing).

With increasing operating time of the multielement oxide catalyst, therefore, there is a decrease not just in the proportion of the molybdenum component in the catalyst but also in the activity of the catalyst in respect of the reaction to be catalysed. This is manifested in a reduced conversion in respect of the component to be oxidized, for example propene. The decline in conversion can be counteracted by increasing the temperature of the salt bath surrounding the tubes of the reactor that contain the catalyst. In this case, the temperature of the salt bath required for a specific conversion is increased to a value which, given otherwise unchanged reaction conditions, achieves the same conversion for the compound to be oxidized in a single pass through the reactor as before the decrease in the activity of the catalyst. However, the increase in the temperature of the salt bath in order to counteract the deactivation of the active composition of a mixed oxide catalyst containing molybdenum in oxidized form is counterproductive in respect of the lifetime of the mixed oxide catalyst in question. This is because higher temperatures in the fixed catalyst bed accelerate the ageing or deactivation of the catalyst, which prematurely necessitates either the full or at least the partial replacement of the aged fixed catalyst bed with fresh catalyst. This inevitably leads to more frequent changes of the fixed catalyst bed and more frequent production shutdowns. As well as the associated loss of production, the more frequent disposal of the spent catalyst and the more frequent production of new catalyst also constitute a considerable financial burden.

As an alternative to the standard preparation processes, mixed oxide catalysts based on bismuth molybdates can also be prepared by means of hydrothermal synthesis. By comparison with the aforementioned preparation processes, the reaction conditions in the hydrothermal synthesis are generally much less severe. Hydrothermal synthesis therefore enables a practical access route of good reproducibility to materials having high purity, controlled morphology and high crystallinity. To date, however, only bismuth molybdates, i.e. only binary systems, have been prepared by means of hydrothermal synthesis. Even though good selectivities for the formation of acrolein in the catalysed partial gas phase oxidation of propene are achieved with bismuth molybdates, these systems are nevertheless unsuitable for use on the industrial scale. This is because the propene conversions achieved with bismuth molybdates in this reaction are not more than about 15%. In the case of such low propene conversions, however, it is then necessary to recycle a particularly large amount of unconverted propene. This results in an immense rise both in the energy expenditure and in the capital costs, which means that an implementation on the industrial scale is of little interest.

Moreover, mixed oxide catalysts used in industry for the oxidation of propene also contain elements other than molybdenum and bismuth. If the hydrothermal synthesis of a mixed oxide catalyst is switched from a binary system such as bismuth molybdate to a more complex multicomponent system containing cobalt and iron as well as bismuth and molybdenum, this has a direct effect on what phases are formed, what amounts of catalytically active elements are incorporated into these phases, and the size of the surface area of the phases.

Since a multielement oxide catalyst optimized in terms of activity and selectivity constitutes a combination of an elevated surface area, suitable crystal phases and a crucial amount of catalytically active elements, there was therefore a need for a process for providing corresponding multielement oxide catalysts with high crystallinity.

This problem is solved in accordance with the invention by conducting a hydrothermal synthesis with an aqueous solution and/or an aqueous suspension of at least four metal precursor compounds, especially at least four transition metal precursor compounds, of the metals present in the multielement oxide catalyst to be prepared, the pH of which has been adjusted to a value between about 6 and about 8.

The present invention therefore provides a process for preparing a multielement oxide catalyst of the general formula (I)

MoₐBi_{b}Co_{c}Fe_{d}NiₑX_{f}X′_{g}X″ₕX′″ᵢX″″ⱼOₓ (I) with

X=W and/or P,

X′=Li, Na K, Rb, Cs, Mg, Ca, Sr and/or Ba,

X″=Ce, Mn, Cr and/or V,

X″′=Nb, Se, Te, Sm, Gd, La, Y, Pd, Pt, Ru, Ag and/or Au,

X″″=Si, Al, Ti and/or Zr,

and

a=12,

b=1 to 4,

c=4 to 10,

d=1 to 4,

e=0 to 4,

f=0 to 5,

g=0 to 2,

h=0 to 5,

i=0 to 2,

j=0 to 800,

and

x=a number which is determined by the valency and frequency of the elements other than oxygen, comprising the steps of
a) providing a mixture of an aqueous solution and/or an aqueous suspension of precursor compounds of the elements present in the multielement oxide catalyst to be prepared in the amount required to achieve the stoichiometry according to the general formula (I),
b) setting the pH of the mixture obtained from step a) to a value between 6+/−0.5 and 8+/−0.5,
c) reacting the precursor compounds containing mixture of step b) under solvothermal reaction conditions in an autoclave at a temperature of more than 100° C. to 600° C. to form a multielement oxide catalyst of the general formula (I), and
d) separating the multielement oxide catalyst from the aqueous solution and/or suspension.

In the context of the present invention, the terms “multielement oxide catalyst” and “mixed oxide catalyst” are used synonymously and refer to a catalyst consisting at least of the elements molybdenum, bismuth, cobalt and iron according to the invention.

The process according to the invention therefore preferably serves for preparation of a multielement oxide catalyst of the general formula (I) BiₐMo_{b}Co_{c}Fe_{d}Oₓ with a=1 to 4, b=12, c=4 to 10 and d=1 to 4.

More particularly, the process according to the invention affords a multielement oxide catalyst of the general formula (I) BiₐMo_{b}Co_{c}Fe_{d}Oₓ with the definitions a=1 to 2, b=12, c=5 to 8 and d=2 to 3.

In the context of the present invention, the term “hydrothermal synthesis” is used in accordance with the common knowledge of the person skilled in the art and refers to a heterogeneous reaction conducted in water at a temperature above 100° C. and a pressure above 1 bar. These reaction conditions, i.e. temperatures and pressures above 100° C. and above 1 bar, are also referred to as solvothermal or hydrothermal reaction conditions. In the case of hydrothermal syntheses, it is generally necessary to use autoclaves. They serve for protection of the reaction vessels, the autoclave itself frequently being the reaction vessel. The pressure in the reaction vessel is regulated via the temperature—when reaction medium is used, temperatures of 200° C. in a reaction vessel isolated from the outside already lead to gauge pressures of 800 bar or more. The use of an autoclave itself as pressure-resistant reaction vessel enables performance of the hydrothermal synthesis over a wide pressure and/or temperature range. Moreover, an autoclave has the advantage over other reaction vessels isolated from the outside, for example ampoules, that it is also possible to continuously supply further readily soluble components to the reaction medium during the reaction, for example acids, bases or other complex-forming substances, which are also referred to as mineralizers. Therefore, the process according to the invention or at least step c) of the process according to the invention is conducted in an autoclave.

With regard to the precursor compounds of the elements present in the multielement oxide catalyst to be prepared, the process according to the invention is not subject to any restrictions in principle. Advantageously, precursor compounds used are those whose anions can be removed without residue in any thermal treatment that follows the hydrothermal synthesis. Preferably, therefore, salts are used as precursor compounds of the elements present in the multielement oxide catalyst to be prepared in step a) of the process according to the invention. In particular, nitrates, carbonates, formates, oxalates or similar compounds are used as precursor compounds in the process according to the invention.

In an embodiment of the process according to the invention, therefore, salts are used as precursor compounds in step a).

In the context of the present invention, the expression “a pH between 6+/−0.5 and 8+/−0.5” encompasses all pH values from 5.5 to 8.5 inclusive which can be expressed by whole and/or real numbers, especially the pH values of 5.5; 6; 6.5; 7; 7.5; 8 and 8.5.

Multielement catalysts which have been prepared at these pH values by means of hydrothermal synthesis catalyse the oxidation of propene to acrolein with a selectivity of at least 50% up to about 82%.

Preferably, the pH in step b) of the process according to the invention is adjusted to a value between 6 and 8, i.e. one which satisfies the condition 6 pH 8, especially to a value greater than 6 and less than 8.

By conducting step b) of the process according to the invention in an even narrower pH range, especially at a pH of 7+/−0.5, not only is a catalyst with a selectivity for the formation of acrolein at values of at least 65% up to about 82% achieved, but the propene conversion is also increased to values of up to about 65%.

In one embodiment of the process according to the invention, therefore, a pH of 7+/−0.5 is established in step b).

In the context of the present invention, it has been found that the choice of pH in the synthesis significantly influences the composition of the catalyst. Rising pH values in the hydrothermal synthesis tend to result in a distinct decrease in the content of the catalytically active molybdenum component in the catalyst prepared; at the same time, a distinct increase in the proportion of the bismuth component is observed. The specialist literature generally recognizes the role of molybdenum as catalytically active component for the gas phase partial oxidation. It should therefore be expected that the catalyst prepared at pH=5 with the highest molybdenum content should also have the best catalytic properties in the partial gas phase oxidation of propene. However, studies have shown that the catalyst according to the invention prepared at a pH of 7+/−0.5 has both the highest acrolein selectivity and the highest propene conversion.

Moreover, it has been found that the pH established in step b) of the process according to the invention also influences the specific surface area of the catalysts: with rising pH, the specific surface area of the catalysts also increases. A high surface area of a catalyst is typically correlated with a high catalytic activity. It should therefore be expected that the catalysts prepared at higher pH values, pH=8 or more, should exhibit the highest propene conversions in the partial gas phase oxidation. However, it has been found in studies that, surprisingly, the catalyst prepared at a pH of 7+/−0.5 exhibits the highest propene conversion in the partial gas phase oxidation of propene.

Moreover, it has been found that the exothermicity in the catalysed reaction for the multielement oxide catalysts prepared at pH=7 is lower than in the case of the comparative catalyst prepared by coprecipitation. Consequently, a corresponding catalyst, when used in the partial gas phase oxidation of olefins, is subject to lower thermal stress. As a result, the catalyst is, for example, not as quickly depleted of the catalytically active molybdenum component. Overall, the lower thermal stress has a positive effect on the lifetime of the catalyst.

A pH of 7+/−0.5 therefore constitutes a particularly favourable choice for the pH in the hydrothermal synthesis. The setting of the pH to 7+/−0.5 seems to lead to a catalyst having a particularly good combination of molybdenum content and specific surface area, since the catalyst obtained under these conditions leads to particularly good acrolein selectivity and simultaneously also to a particularly good propene conversion.

It may be the case that the precursor compounds of the elements present in the catalyst to be prepared do not attain the requisite solubility in pure water as solvothermal reaction medium. For formation of other complexes of the elements in question that have better solubility than with pure water, therefore, preference is given to adding an acid, base or another complex-forming substance to the solution and/or suspension of the precursor compounds. With regard to the acid, base or complex-forming substance to be added, the process according to the invention is not subject to any restrictions in principle. Advantageously, the acid, base or complex-forming substance to be added is chosen such that it can be removed without residue with a low degree of complexity after the hydrothermal synthesis, for example by simple washing or by breakdown at elevated drying temperatures. Auxiliaries used in the hydrothermal synthesis, also referred to as mineralizers, are therefore, for example, nitric acid, acetic acid, formic acid or similar compounds. Preferably, in step a) of the process according to the invention, nitric acid is added to improve the solubility of the precursor compounds.

In addition to the aforementioned acids, some of the precursor compounds and/or some of the metal cations of the precursor compounds in hydrated form behave as acids, in some cases as strong acids, in an aqueous medium. By adding a suitable base, the desired pH is established in step b) of the process according to the invention. With regard to the choice of this base, the process according to the invention is not subject to any restrictions in principle, provided that it is assured that it is possible by the addition thereof to establish a pH between 6+/−0.5 and 8+/−0.5, preferably a pH greater than 6 and less than 8 and especially a pH of 7+/−0.5, and the added base does not disrupt the formation of the multielement oxide catalyst. Preferably, in the process according to the invention, the desired pH is established by using a base which can be removed easily from the catalyst prepared by washing operations and/or by a subsequent thermal treatment, especially at low temperatures. Preference is given to using a complexing base for establishment of the pH in step b) of the process according to the invention. Without wishing to be bound to a specific theory, it is believed that the presence of a complexing base has an advantageous effect on the catalytic properties of the multielement oxide catalyst prepared. Therefore, preference is given to using a nitrogen base for establishment of the pH in step b) of the process according to the invention. Preferably, the nitrogen base is ammonia or a primary, secondary or tertiary aliphatic C₁-C₄-amine, for example methylamine, ethylamine, dimethylamine, diethylamine, trimethylamine or triethylamine. Preference is given to using an aqueous ammonia solution as base, especially an aqueous ammonia solution having a concentration of at least 20%, especially having a concentration of 20% to 30%, for example 25%.

The reaction vessel, preferably the autoclave, is positioned in an oven for the performance of the hydrothermal synthesis: it is possible to work either isothermally or with a temperature gradient. Preference is given to working isothermally in the context of the process according to the invention, meaning that the filled autoclave together with its contents, after sealing, is heated up to a defined temperature, this temperature is maintained for a specific period of time and, thereafter, the autoclave together with its contents is cooled down over a specific period of time to the starting temperature, preferably room temperature, or the autoclave together with its contents is allowed to cool down to the starting temperature, preferably room temperature. This procedure enables performance of the process according to the invention under essentially constant reaction conditions.

According to the invention, step c) is conducted at a temperature of more than 100° C. to 600° C. Based on water as solvothermal reaction medium, the performance of step c) at these temperatures corresponds to an internal pressure in the autoclave of more than 1 bar up to about 3000 bar.

In an embodiment of the process according to the invention, step c) is conducted at a temperature of more than 100° C. to 400° C. This reaction temperature leads, in the case of performance of step c) with water as solvothermal reaction medium, to an internal pressure of more than 1 bar up to about 100 bar.

In the context of the present invention, the statement “a temperature of more than 100° C. to 600° C.” and the statement “a temperature of more than 100° C. to 400° C.” encompass all values of, respectively, more than 100° C. up to and including 600° C. and of more than 100° C. up to and including 400° C. which can be expressed by whole and real numbers.

In the context of the present invention, the statement “a pressure of more than 1 bar to 3000 bar” and the statement “a pressure of more than 1 bar to 100 bar” encompass all values of, respectively, more than 1 bar up to and including 3000 bar and of more than 1 bar up to and including 100 bar which can be expressed by whole and real numbers.

With regard to the period of time within which step c) is conducted, the process according to the invention is not subject to any restrictions, provided that it is still assured that the process gives a mixed oxide catalyst of the composition according to the invention.

In one embodiment of the process according to the invention, therefore, step c) is conducted for a period of 6+/−0.5 hours up to 48+/−0.5 hours.

Advantageously, the process according to the invention is followed by further steps for aftertreatment of the multielement oxide catalyst obtained from the process according to the invention. More particularly, these steps involve single or multiple, preferably multiple, washing of the multielement oxide catalyst obtained from the hydrothermal synthesis for removal of residues from the synthesis. Suitable solvents for washing are in principle all of those which can fully remove residues from the synthesis in one or more washing operations and leave the synthesized catalyst unchanged in terms of its structure and composition. Preference is given, however, to those solvents which can themselves also be removed from the catalyst without residue with a low degree of complexity. Particularly those solvents removable by drying at low temperatures, especially at temperatures below the 400° C. customary for the calcination, are used with preference for washing. Illustrative solvents for washing are water and acetone. The multielement oxide catalyst obtained from the synthesis is then, for example, washed once or more than once, preferably more than once, for example three times, with water and then once or more than once, preferably more than once, for example three times, with acetone. Thereafter, the washed multielement oxide catalyst is dried. This is preferably done at temperatures below the temperatures of 400° C. or more that are customary for a calcination. Preferably, the temperature in the drying of the multielement oxide catalyst ranges from 20° C. to a maximum of 390° C.; the drying temperature preferably ranges from 20° C. to 350° C. These temperatures, which are below the temperatures typically employed for a calcination, are already sufficient for nitrates to break down. The duration of the drying is chosen to be sufficiently long in order to ensure that all residues from the synthesis and all solvent from the washing operation have been fully removed from the multielement oxide catalyst. The juncture at which this has been achieved can be determined by drying until the occurrence of constant weight, for example by means of thermogravimetry. A duration of up to 3 days, especially 12, 24, 36 or 48 hours, is generally sufficient, even at a temperature of 20° C., for drying of a washed multielement oxide catalyst. By contrast with the multielement oxide catalysts obtained by the existing synthesis methods, especially coprecipitation, calcination of the multielement oxide catalyst obtained from the process according to the invention is not required in principle. According to the invention, therefore, the preparation of multielement oxide catalysts is effected under much milder conditions than in the coprecipitation. By contrast with this standard preparation method, in the process according to the invention, enrichment of bismuth in the catalytically active phase is also avoided. Optionally, as an alternative or in addition to drying, it is nevertheless possible to conduct a calcination in the process according to the invention.

In one embodiment of the process according to the invention, the process therefore additionally comprises the steps of
e) washing the multielement oxide catalyst obtained from step d) at least once, and
f) then drying and/or calcining the multielement oxide catalyst.

The multielement oxide catalyst obtained by the process according to the invention is typically in powder form. For industrial use, it is advantageous to convert the pulverulent catalyst, after addition of shaping agents and binders, to a better manageable form. This can be effected by applying the catalyst to a support, for example composed of aluminium oxide, zirconium oxide, titanium dioxide or silicon dioxide, or by other shaping steps such as tableting or extrusion. The geometric shape of the support is not limiting here, but instead is guided by the specifications of the reactor, for example tube diameter, length of the catalyst bed. The support may therefore, for example, be a pyramid, a cylinder, a saddle, a ball or a polygon, but it may also be a wall of a reaction space, for example in the form of a wall reactor.

Binders used may be various oils, celluloses, polyvinyl alcohols, saccharides, acrylates, alkyl derivatives thereof, condensates thereof or mixtures thereof. Preferred binders are acrylates, polyvinyl alcohols, cellulose, alkyl derivatives thereof and mixtures thereof.

A supported catalyst is prepared, for example, by spraying either a suspension of the catalyst in a suitable solvent or solvent mixture together with the binder onto the support body or by spraying a suspension of the catalyst onto a support body moistened with the binder. Suitable solvents are, for example, water, methanol, ethanol or similar compounds or mixtures thereof. An unsupported catalyst is prepared, for example, by mixing the catalyst with an aforementioned binder and subsequent shaping.

The catalyst obtained by the respective shaping operation is subsequently subjected to a drying and/or calcination to complete the removal of the solvents and especially of the binders.

In a further embodiment, the process according to the invention therefore comprises the additional steps of
i) applying the multielement oxide catalyst obtained from step d), e) or f) to a support to obtain a supported catalyst,

or
ii) forming the multielement oxide catalyst obtained from step d), e) or f) to obtain an unsupported catalyst,

and
iii) drying and/or calcining the multielement oxide catalyst obtained from step i) or ii).

Studies of the multielement oxide catalysts obtained by the process according to the invention in a scanning electron microscope with energy-dispersive x-ray spectroscopy (SEM-EDX) have shown that the catalysts have areas in which both molybdenum and bismuth, which are considered to be the catalytically active elements, and iron are simultaneously present together. It is believed that the improved catalytic properties of the catalysts according to the invention in the partial gas phase oxidation of olefins are attributable to the joint presence of the elements molybdenum, bismuth and iron in areas of the catalysts.

The present invention therefore also further provides a multielement oxide catalyst of the general formula

MoₐBi_{b}Co_{c}Fe_{d}NiₑX_{f}X′_{g}X″ₕX′″ᵢX″″ⱼOₓ (I)

with

X=W and/or P,

X′=Li, K, Na, Rb, Cs, Mg, Ca, Ba and/or Sr,

X″=Ce, Mn, Cr and/or V,

X″′=Nb, Se, Te, Sm, Gd, La, Y, Pd, Pt, Ru, Ag and/or Au,

X″′=Si, Al, Ti and/or Zr,

and

a=12,

b=1 to 4,

c=4 to 10,

d=1 to 4,

e=0 to 4,

f=0 to 5,

g=0 to 2,

h=0 to 5,

i=0 to 2,

j=0 to 800,

and

x=a number which is determined by the valency and frequency of the elements other than oxygen,

which is characterized in that it comprises areas in which molybdenum, bismuth and iron are simultaneously present, and said areas have a diameter of from 10 nm to 25 μm.

By comparison a common multielement oxide catalyst according to the prior art, that contains said three elements as well, does not have any areas in which all of the three elements are simultaneously present. Rather, a common multielement oxide catalyst has areas in which only one of the elements is present.

In one embodiment of the multielement oxide catalyst according to the invention the areas, in which molybdenum, bismuth and iron are simultaneously present, have a diameter of from 1 μm to 22 μm.

In one embodiment, the multielement oxide catalysts according to the invention are obtained and/or obtainable by the process according to the invention.

In that case, the elements molybdenum, bismuth and iron are especially present together in regions or zones of the catalyst according to the invention when the pH in the hydrothermal synthesis of the catalysts is 7+/−0.5. In this case, a pH of 7+/−0.5 is established in step b) of the process according to the invention.

In one embodiment, the multielement oxide catalyst according to the invention is obtained and/or obtainable by the process according to the invention, wherein a pH of 7+/−0.5 is established in step b) of the process according to the invention.

In one embodiment of the multielement oxide catalyst according to the invention, e to i are each 0.

In a further embodiment of the multielement oxide catalyst according to the invention, a=12, b=1 to 2, c=5 to 8 and d=2 to 3.

By contrast with the multielement oxide catalysts obtained by standard preparation processes, especially those obtained by coprecipitation, the multielement oxide catalysts obtainable and/or obtained by the process according to the invention need not be subjected to a calcination; instead, drying conducted at lower temperatures than those employed in a calcination are already sufficient to bring the catalyst into the form required for use in catalysis. Calcination and use in catalysis generally affect the phases of the catalyst in question, such that the catalysts used in catalysis is typically no longer identical to the original catalyst obtained from the standard preparation process or the catalyst present at the start of the catalysis. It has now been found that, surprisingly, the catalyst according to the invention and especially the multielement oxide catalyst obtained and/or obtainable by the process according to the invention are essentially phase-stable. This means that these multielement oxide catalysts are subject to a low degree of phase transformation, if any at all, during the catalysed reaction. The low degree of phase transformation consists in an intensity gain for the Raman signal at 875 cm⁻¹ (FeMoO₄) during the catalytic reaction. By contrast, a comparative catalyst obtained by the preparation process of coprecipitation, for example the catalyst Mo₁₂Bi_{1.5}Co₅Fe_{1.8}Oₓ obtained by the process of WO 2007/042369 A1, is subject to significant phase reformation during the catalysed reaction: The Raman signal for MoO₃ at 995 cm⁻¹ disappears, the Raman signals for Bi₂Mo₃O₁₂ at 814 cm⁻¹ and 900 cm⁻¹ weaken, and the

Raman signal for FeMoO₄ at 875 cm⁻¹ appears.

In one embodiment, the multielement oxide catalyst according to the invention, especially the multielement oxide catalyst obtained and/or obtainable by the process according to the invention, is therefore essentially phase-stable, especially during use in the partial gas phase oxidation of olefins or tert-butanol.

The multielement oxide catalysts of the prior art, in particular those prepared by coprecipitation, contain a MoO₃-phase. Said phase is present in the coprecipitaed catalyst before or after their use in the partial gas phase oxidation or ammoxidation. By comparison the catalysts according to the invention, especially the catalysts obtained and/or obtainable by the process according to the invention, do not contain a MoO₃-phase, before their use in a partial gas phase oxidation or in an ammoxidation reaction as well as after their use in a partial gas phase oxidation or in an ammoxidation reaction. However, the catalysts according to the invention and the catalysts obtained and/or obtainable by the process according to the invention contain an alpha-bismuth molybdate phase and a beta-cobalt molybdate phase.

In an embodiment, the multielement oxide catalysts according to the invention, especially the multielement oxide catalysts obtained and/or obtainable by the process according to the invention, do not contain a MoO₃-phase.

In one embodiment, the multielement oxide catalysts according to the invention, especially the multielement oxide catalysts obtained and/or obtainable by the process according to the invention, have an alpha-bismuth molybdate phase and a beta-cobalt molybdate phase.

An alpha-bismuth molybdate phase and a beta-cobalt molybdate phase are possessed especially by the multielement oxide catalysts obtained and/or obtainable by the process according to the invention that have been obtained at a pH of 7+/−0.5 in step b) of the process according to the invention.

The multielement oxide catalysts obtained and/or obtainable by the process according to the invention and the multielement oxide catalysts according to the invention are suitable for the partial gas phase oxidation and ammoxidation of olefins or tert-butanol.

In the context of the present invention, the terms “partial oxidation”, “partial gas phase oxidation” and “gas phase partial oxidation” are used in such a way that they refer to conversions of organic compounds under the reactive action of oxygen in which the compound to be oxidized, after the reaction has ended, contains at least one chemically bonded oxygen atom more than before. By contrast with a full oxidation, a partial oxidation, a partial gas phase oxidation or a gas phase partial oxidation does not oxidize all the carbon atoms present in the compounds to be oxidized to carbon dioxide.

The present invention therefore also further provides for the use of a multielement oxide catalyst obtained and/or obtainable by the process according to the invention and/or of a multielement oxide catalyst according to the invention in the partial gas phase oxidation and/or in the ammoxidation of olefins or tert-butanol.

The partial gas phase oxidation of olefins leads to unsaturated aldehydes and the corresponding unsaturated carboxylic acids, and the ammoxidation of olefins leads to unsaturated nitriles. The most economically important unsaturated aldehydes, carboxylic acids and nitriles are C₃ and C₄ compounds. In the context of the present invention, therefore, the mixed oxide catalysts obtained and/or obtainable by the process according to the invention and/or the mixed oxide catalysts according to the invention are used in the industrial preparation of acrolein and acrylic acid by catalysed gas phase partial oxidation of propene, methylacrolein and methacrylic acid by catalysed gas phase partial oxidation of isobutene (2-methyl-1-propene) or tert-butanol (2-methyl-2-propanol) and the industrial preparation of acrylonitrile by ammoxidation of propene. The preparation of these compounds is effected in the form of heterogeneously catalysed oxidation of propene or of isobutene or tert-butanol with air or oxygen or in the form of heterogeneously catalysed ammoxidation of propene with ammonia and air or oxygen over a fixed catalyst bed comprising predominantly a mixed oxide catalyst based on molybdenum oxides and bismuth oxides.

In one embodiment of the use according to the invention, therefore, the olefin is propene and/or isobutene.

In the context of the present invention, it has been found that the multielement oxide catalysts obtained and/or obtainable by the process according to the invention and/or the multielement oxide catalysts according to the invention are much more selective for the formation of unsaturated aldehydes than for the formation of unsaturated carboxylic acids.

Preferably, therefore, the use according to the invention is the partial gas phase oxidation of olefins to unsaturated aldehydes. More particularly, the use according to the invention is the partial gas phase oxidation of propene to acrolein and/or the partial gas phase oxidation of isobutene and/or tert-butanol to methacrolein.

The present invention is further described by the following items:

1. Process for preparing a multielement oxide catalyst of the general formula (I)

MoₐBi_{b}Co_{c}Fe_{d}NiₑX_{f}X′_{g}X″ₕX′″ᵢX″″ⱼOₓ (I) with
X=W and/or P,
X′=Li, K, Na, Rb, Cs, Mg, Ca, Ba and/or Sr,
X″=Ce, Mn, Cr and/or V,
X″′=Nb, Se, Te, Sm, Gd, La, Y, Pd, Pt, Ru, Ag and/or Au,
X″″=Si, Al, Ti and/or Zr,
and
a=12,
b=1 to 4,
c=4 to 10,
d=1 to 4,
e=0 to 4,
f=0 to 5,
g=0 to 2,
h=0 to 5,
i=0 to 2,
j=0 to 800,
and
x=a number which is determined by the valency and frequency of the elements other than oxygen, comprising the steps of
a) providing a mixture of an aqueous solution and/or an aqueous suspension of precursor compounds of the elements present in the multielement oxide catalyst to be prepared in the amount required to achieve the stoichiometry according to the general formula (I),
b) setting the pH of the mixture obtained from step a) to a value between 6+/−0.5 and 8+/−0.5,
c) reacting the precursor compounds containing mixture of step b) under solvothermal reaction conditions in an autoclave at a temperature of more than 100° C. to 600° C. to form a multielement oxide catalyst of the general formula (I), and
d) separating the multielement oxide catalyst from the aqueous solution and/or suspension.

2. Process according to item 1, wherein salts are used as precursor compounds in step a).

3. Process according to item 1 or 2, wherein a pH of 7+/−0.5 is established in step b).

4. Process according to any one of items 1 to 3, wherein step c) is conducted at a temperature of more than 100° C. to 400° C.

5. Process according to any one of items 1 to 4, wherein step c) is conducted for a period of 6+/−0.5 hours up to 48+/−0.5 hours.

6. Process according to any one of items 1 to 5, additionally comprising the steps of
e) washing the multielement oxide catalyst obtained from step d) at least once, and
f) then drying and/or calcining the multielement oxide catalyst.

7. Process according to any one of items 1 to 6, additionally comprising the steps of
i) applying the multielement oxide catalyst obtained from step d), e) or f) to a support to obtain a supported catalyst,
or
ii) forming the multielement oxide catalyst obtained from step d), e) or f) to obtain an unsupported catalyst,
and
ii) drying and/or calcining the multielement oxide catalyst obtained from step i) or ii).

8. Multielement oxide catalyst of the general formula

MoₐBi_{b}Co_{c}Fe_{d}NiₑX_{f}X′_{g}X″ₕX′″ᵢX″″ⱼOₓ (I)
with
X=W and/or P,
X′=Li, K, Na, Rb, Cs, Mg, Ca, Ba and/or Sr,
X″=Ce, Mn, Cr and/or V,
X″′=Nb, Se, Te, Sm, Gd, La, Y, Pd, Pt, Ru, Ag and/or Au,
X″″=Si, Al, Ti and/or Zr,
and
a=12,
b=1 to 4,
c=4 to 10,
d=1 to 4,
e=0 to 4,
f=0 to 5,
g=0 to 2,
h=0 to 5,
i=0 to 2,
j=0 to 800,
and
x=a number which is determined by the valency and frequency of the elements other than oxygen,
which is characterized in that it comprises areas, in which molybdenum, bismuth and iron are simultaneously present, and said areas have a diameter of from 10 nm to 25 μm.

9. Multielement oxide catalyst according to item 8, obtained and/or obtainable by a process according to any one of claims 1 to 7, wherein a pH of 7+/−0.5 is set in step b) of the process.

10. Multielement oxide catalyst according to item 8 or 9, wherein e to i are each 0.

11. Multielement oxide catalyst according to any one of items 8 to 10, wherein a=12, b=1 to 2, c=5 to 8 and d=2 to 3.

12. Multielement oxide catalyst according to any one of items 8 to 11, wherein the catalyst does not contain a MoO₃-phase.

13. Multielement oxide catalyst according to any one of items 8 to 12, wherein the multielement oxide catalyst has an alpha-bismuth molybdate phase and a beta-cobalt molybdate phase.

14. Use of a multielement oxide catalyst obtained and/or obtainable by a process according to any one of items 1 to 7 and/or of a multielement oxide catalyst according to any one of items 8 to 13 in the partial gas phase oxidation and/or in the ammoxidation of olefins or tert-butanol.

15. Use according to item 14, wherein the olefin is propene and/or isobutene.

### DESCRIPTION OF THE FIGURES

FIG. 1: Correlation between the pH and the composition of the catalysts of experiments 1 to 3 and the comparative examples C1 to C3.

FIG. 2: Correlation between the pH and the composition of the catalysts of experiments 4 to 6.

FIG. 3: BET surface area of the Mo₁₂Bi₁Co₈Fe₃Oₓ catalyst prepared hydrothermally as a function of the pH in the hydrothermal synthesis and the drying temperature prior to the BET measurement (●=drying at 320°; ▪=drying at 150° C., wherein the temperatures are the drying temperatures prior to the BET measurements).

FIG. 4: SEM-EDX image of the multielement catalyst from experiment 1 (the elements bismuth Bi, molybdenum Mo and iron Fe are coloured as stated in the image).

FIG. 5: SEM-EDX image of the multielement catalyst from experiment 2 (the elements bismuth Bi, molybdenum Mo and iron Fe are coloured as stated in the image).

FIG. 6: SEM-EDX image of the multielement catalyst from experiment 3 (the elements bismuth Bi, molybdenum Mo and iron Fe are coloured as stated in the image).

FIG. 7: Phase transformations of the catalysts prepared by hydrothermal synthesis at pH=6, 7 or 8 of experiments 1 to 3 (indicated as pH=6, pH=7 and pH=8 in FIG. 7) and the catalyst prepared by coprecipitation according to WO 2007/042369 A1 (indicated as CP in FIG. 7) (Raman spectra before the catalysed reaction (dark line) and thereafter (light line)).

FIG. 8: Phase compositions of the catalyst Mo₁₂Bi_{1.5}Co₅Fe₃Oₓ synthesized hydrothermally at pH values of from 6 to 10.

FIG. 9: Time-dependent phase transformation of the Mo₁₂Bi_{1.5}Co₅Fe_{1.8}Oₓ catalyst obtained by coprecipitation.

FIG. 10: Time-dependent phase transformation of the Mo₁₂Bi_{1.5}Co₅Fe₃Oₓ catalyst synthesized hydrothermally at pH=7.

### EXAMPLES

### Example 1

### Preparation of Mo—Bi—Co—Fe Oxides

A first solution (referred to hereinafter as solution I) was prepared by first dissolving amounts of bismuth(III) nitrate pentahydrate, cobalt(II) nitrate hexahydrate and iron(III) nitrate nonahydrate as defined by the figures in the table in 20 ml of nitric acid (concentration 2 M) and stirring thoroughly for 15 minutes. In parallel, a second solution (referred to hereinafter as solution II) was prepared by dissolving stoichiometric amounts of ammonium heptamolybdate according to the figures in Table 1 in 20 ml of demineralized water and stirring for 15 minutes. In each case, the molar amounts of the precursor compounds of bismuth, molybdenum, cobalt and iron were chosen such that they added up to 20 mmol in total. Solution I and solution II were combined in an autoclave insert made of Teflon®. Thereafter, the pH was adjusted by dropwise addition of a 25% ammonia solution by means of a titrator (Schott Instruments), and the solution obtained was stirred for a further 15 minutes. Thereafter, the autoclave insert together with the solution present therein was transferred into a steel autoclave having a volume of 250 ml (from Bergerhof). Then the autoclave was closed and heated to a temperature of 180° C. for 24 hours. After this period of time, the autoclave together with its contents was allowed to cool down to room temperature over a period of a further 24 hours. Thereafter, the product obtained was filtered through a G4 glass frit (nominal pore size 10 to 16 micrometres). The solid product was washed three times with 10 ml of water and three times with 10 ml of acetone. Finally, the solid product was dried at room temperature for 48 hours and calcined at 320° C. for 5 hours.

The compositions of the catalysts from experiments 1 to 6 and comparative experiments C1 to C3 were determined in Example 3.

TABLE 1
Overview of the hydrothermally synthesized multielement oxide catalysts

| | Molar proportions/% | | | | | | | | | | |
|---|---|---|---|---|---|---|---|---|---|---|---|
| Experiment | Bi | Mo | Co | Fe | pH | n (HNO₃)/mmol | V(NH₃)/ml | Bi(NO₃)₃/g | (NH₄)₆Mo₇O₂₄/g | Co(NO₃)₂/g | Fe(NO₃)₃/g |
| C1 | 7.2 | 59.2 | 24.8 | 8.8 | 5 | 0.4 | 4.3 | 0.695 | 2.092 | 1.445 | 0.709 |
| 1 | 7.2 | 59.2 | 24.8 | 8.8 | 6 | 0.4 | 4.5 | 0.695 | 2.092 | 1.445 | 0.709 |
| 2 | 7.2 | 59.2 | 24.8 | 8.8 | 7 | 0.4 | 5.0 | 0.695 | 2.092 | 1.445 | 0.709 |
| 3 | 7.2 | 59.2 | 24.8 | 8.8 | 8 | 0.4 | 5.9 | 0.695 | 2.092 | 1.445 | 0.709 |
| C2 | 7.2 | 59.2 | 24.8 | 8.8 | 9 | 0.4 | 9.4 | 0.695 | 2.092 | 1.445 | 0.709 |
| C3 | 7.2 | 59.2 | 24.8 | 8.8 | 10 | 0.4 | 10.5 | 0.695 | 2.092 | 1.445 | 0.709 |
| 4 | 4.2 | 50.0 | 33.3 | 12.5 | 6 | 0.4 | 4.4 | 0.404 | 1.766 | 1.940 | 1.010 |
| 5 | 4.2 | 50.0 | 33.3 | 12.5 | 7 | 0.4 | 4.8 | 0.404 | 1.766 | 1.940 | 1.010 |
| 6 | 4.2 | 50.0 | 33.3 | 12.5 | 8 | 0.4 | 5.9 | 0.404 | 1.766 | 1.940 | 1.010 |

### Example 2

### Determination of Surface Area of the Catalysts According to the Invention

By means of the BET analysis, the specific surface area of the Mo₁₂Bi₁Co₈Fe₃Oₓ catalyst prepared by hydrothermal means at different pH values was determined. For this purpose, 100 to 500 mg of the catalyst were dried at 150° C. and at 340° C. under reduced pressure for 5 hours, wherein the temperatures are the drying temperatures prior to the BET measurements. The BET analyses were conducted with a BELSORP-Mini II from Rubotherm GmbH. The purge gas used was helium. After drying, the catalyst sample was evacuated and cooled to a temperature of 77 K with liquid nitrogen. The partial pressure of the nitrogen adsorption gas was increased stepwise, and a standard adsorption isotherm in the range of p/p₀=0 to 1 was recorded. Finally, the desorption isotherm was measured. The BET surface area was determined with the aid of BET theory in the range of p/p₀=0.05 to 0.3.

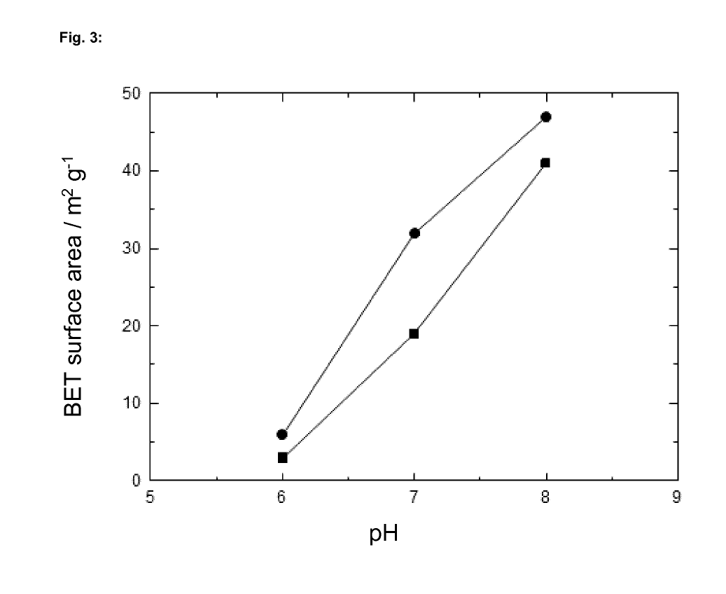

The BET surface areas determined were correlated both with the pH in the synthesis of the particular catalyst and with the drying temperatures. FIG. 3 shows these correlations.

The specific surface area of the catalysts rises with the pH established in the synthesis. Drying conducted at higher temperatures likewise leads to a greater specific surface area.

### Example 3

### Elemental Analysis of the Catalysts Prepared

The composition of the catalysts of experiments 1 to 6 and comparative examples C1 to C3 was conducted by means of optical emission spectroscopy with inductively coupled plasma (ICP-OES, Agilent 720/725-ES). The plasma was generated by a 40 MHz high-frequency generator, and argon was used as plasma gas. The digestion of about 40 mg of sample for the optical emission spectroscopy was effected by suspending the sample in a mixture of 6 ml of concentrated hydrochloric acid, 2 ml of concentrated nitric acid and 1 ml of hydrogen peroxide, and subsequent treatment of the mixture obtained in a microwave at 600 watts for 45 minutes.

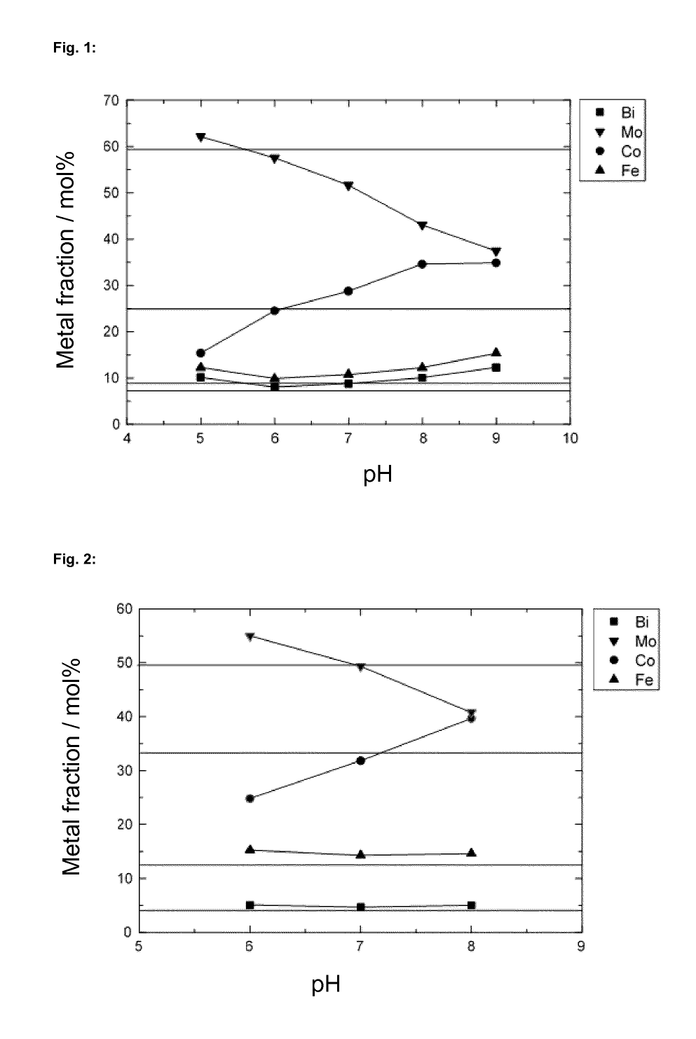

The metal fractions per mole determined for the respective catalysts were correlated with pH values established for the hydrothermal synthesis. These correlations are shown in FIG. 1 for the catalysts of experiments 1 to 3 and comparative experiments C1 to C3 and in FIG. 2 for the catalysts of experiments 4 to 6.

The multielement oxide catalyst prepared at pH=6 from experiment 1 had a composition of roughly Mo₁₂Bi_{1.5}Co₅Fe_{1.8}Oₓ. Proceeding from pH=5, the molar proportion of the catalytic molybdenum component decreases continuously with rising pH from about 62 mol % down to about 37 mol % at pH=9. At the same time, with rising pH, the molar proportion of cobalt increases from 15 mol % at pH=5 up to 35 mol % at pH=9. The proportion of iron and bismuth increases only by a few mol % with rising pH.

The multielement oxide catalyst prepared at pH=7 from example 5 had a composition of roughly Mo₁₂Bi₁Co₈Fe₃Oₓ. Proceeding from pH=6, the molar proportion of the catalytic molybdenum component decreases continuously with rising pH from 55 mol % down to about 40 mol % at pH=8. At the same time, with rising pH, the molar proportion of cobalt increases from 25 mol % up to about 40 mol % at pH=8. The proportions of iron and bismuth remain essentially constant irrespective of the pH at about 5 mol % and about 15 mol % respectively.

The correlation between the pH values in the hydrothermal synthesis and the particular composition of the catalyst shows that the pH has a great influence on the composition of the catalyst. Particularly the molar proportion of the catalytically active molybdenum component is greatly affected by the pH.

### Example 4

### SEM-EDX Analyses

The surface of the multielement oxide catalysts from experiments 1 to 3 was examined in a scanning electron microscope with energy-dispersive x-ray spectroscopy (SEM-EDX).

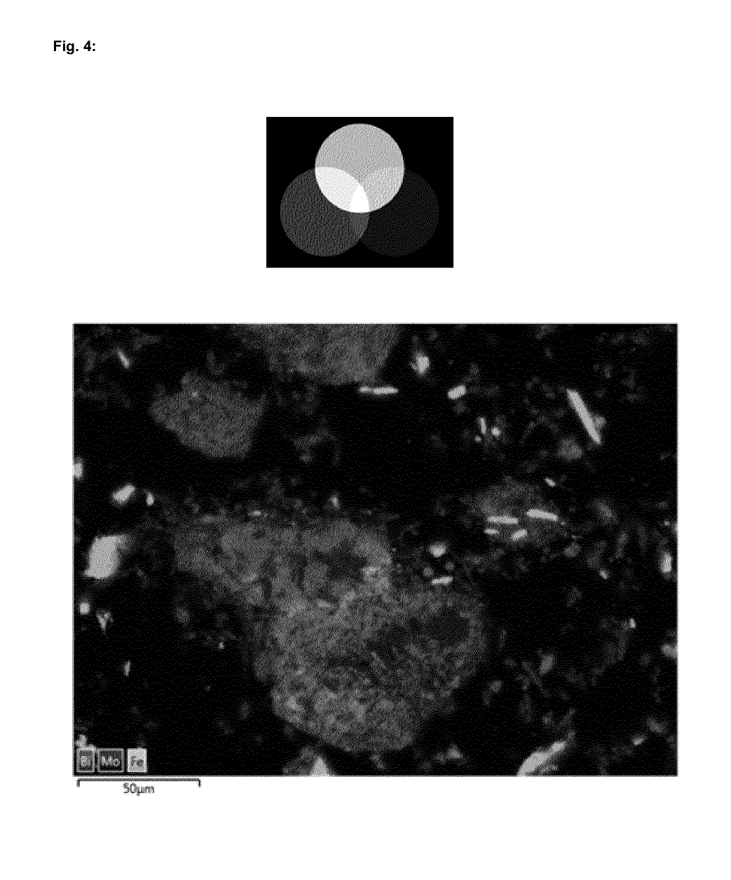

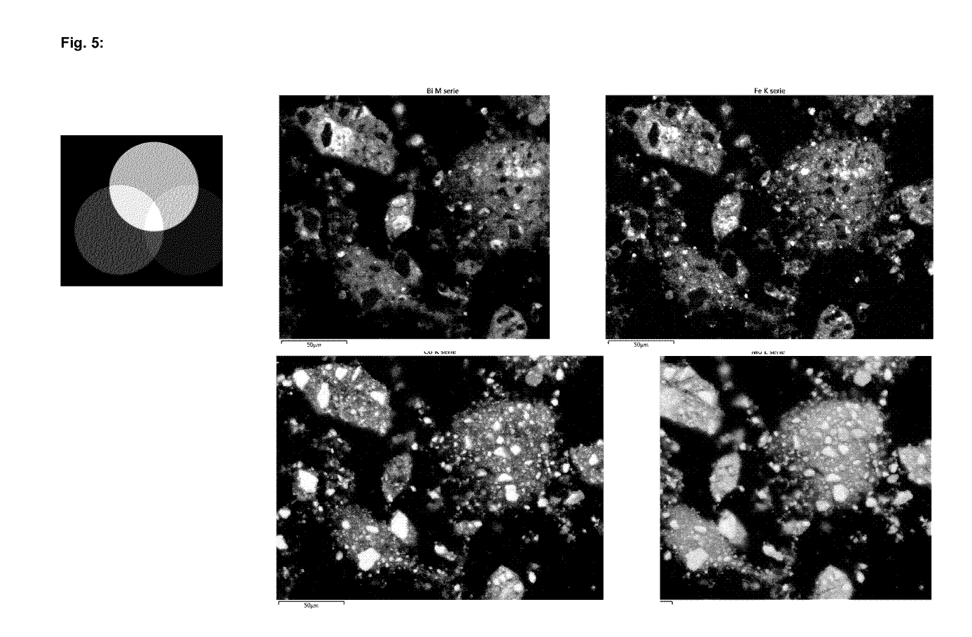

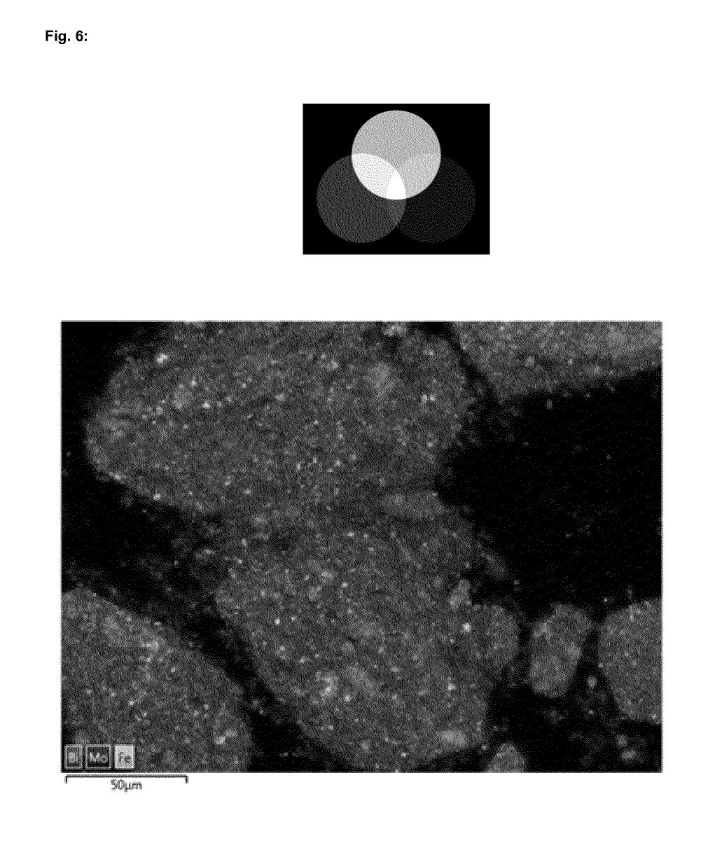

The images of the surfaces of the multielement oxide catalysts of experiments 1 to 3 which have been made by means of SEM-EDX are shown in FIGS. 4 to 6. In these images, the elements molybdenum Mo, bismuth Bi and iron Fe have been coloured. Areas of the catalysts in which all three elements are present together—corresponding to the overlapping of the colours for the individual elements—are shown in white in the figures. Only the multielement oxide catalyst from experiment 2, prepared at pH=7, has white areas, i.e. areas in which all three elements molybdenum, bismuth and iron are present together (see FIG. 5). The diameter of these areas was determined to be in the range of from 10 nm to 25 μm.

### Example 5

### Phase Determinations

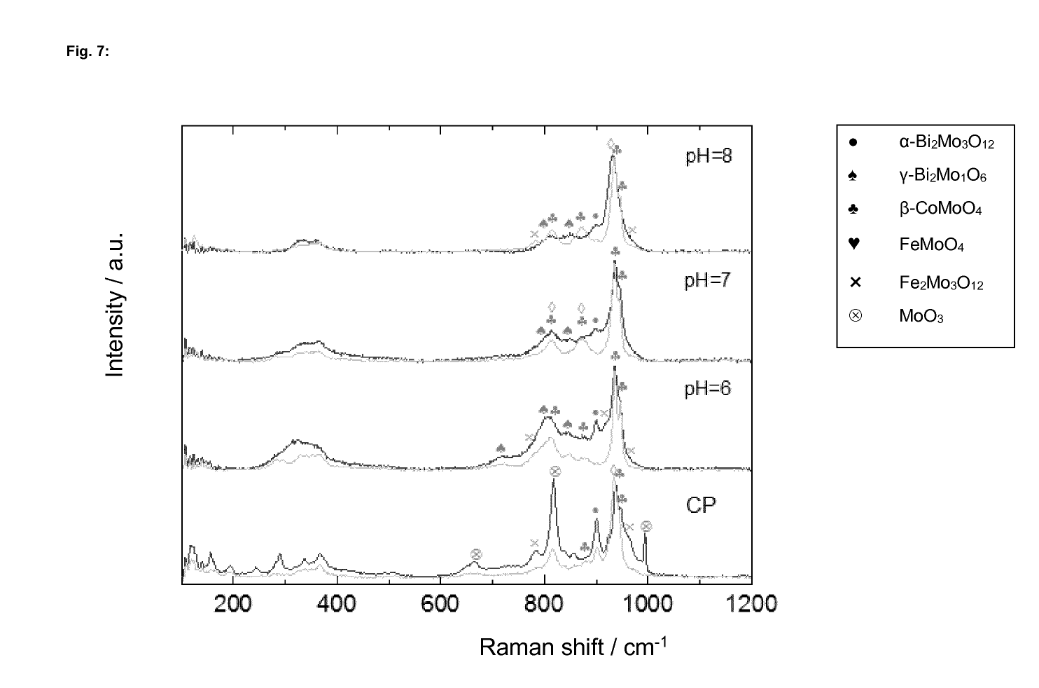

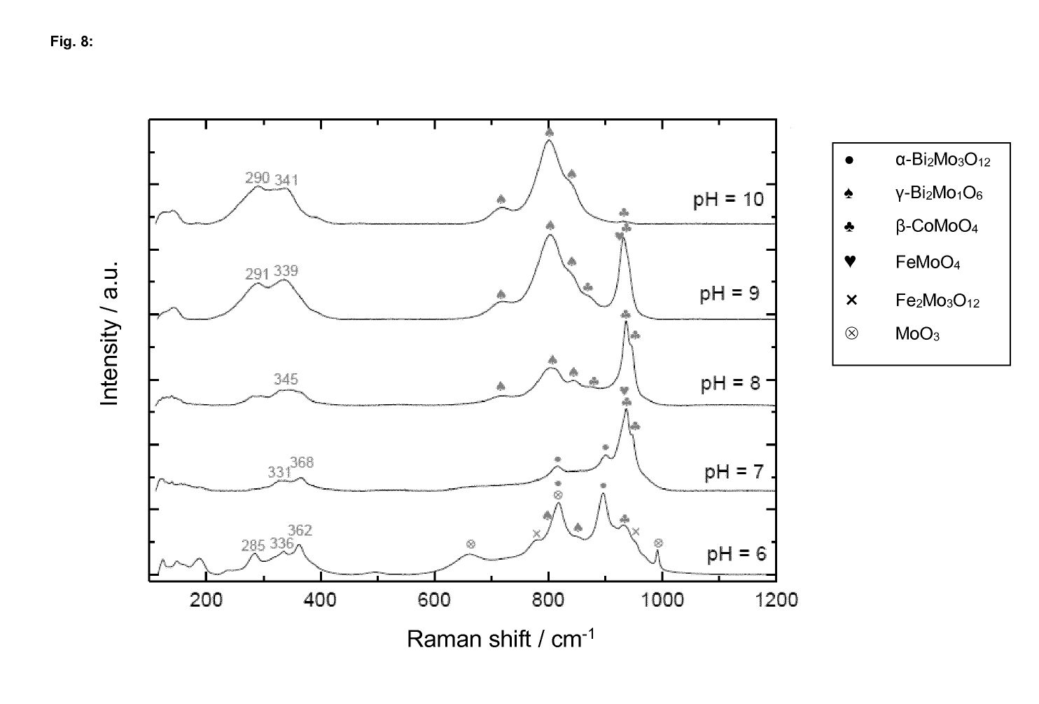

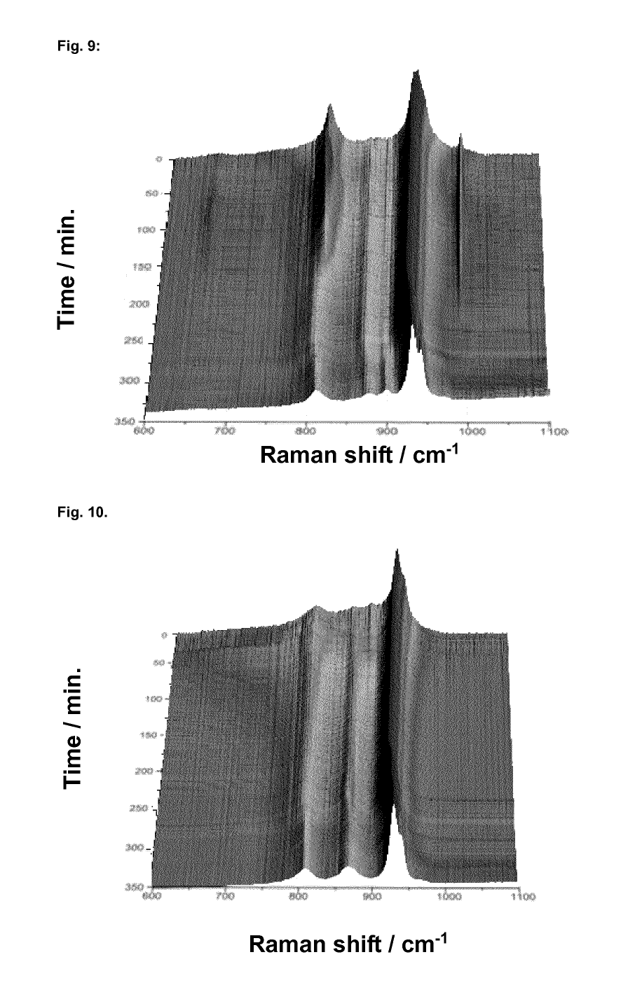

The phases of the catalysts Mo₁₂Bi_{1.5}Co₅Fe₃Oₓ, synthesized hydrothermally at a pH of 6, 7 or 8 and synthesized by coprecipitation (FIG. 7), the catalysts Mo₁₂Bi_{1.5}Co₅Fe₃Oₓ of experiments 1 to 3 and comparative experiments C2 to C3, synthesized hydrothermally at a pH of from 6 to 10 (FIG. 8), the catalyst Mo₁₂Bi_{1 5}Co₅Fe_{1.8}Oₓ, synthesized by coprecipitation (FIG. 9), and the catalyst Mo₁₂Bi_{1.5}Co₅Fe₃Oₓ, synthesized hydrothermally at pH=7 (FIG. 10), were determined by means of Raman spectroscopy. For this purpose, a Renishaw inVia reflex spectrometer system from Renishaw equipped with a research-grade Leica DM2500 microscope was used. The samples were analysed with a 17 mW helium-neon laser at 623 nm. For this purpose, typically the spectral range of 50 to 1300 cm⁻¹ was recorded.

For the recording of FIG. 8 (catalysts Mo₁₂Bi_{1.5}Co₅Fe₃Oₓ of experiments 1 to 3 and comparative experiments C2 to C3, synthesized hydrothermally at a pH of from 6 to 10), the laser beam was focused onto the sample at room temperature with a Leica 50× objective lens. In each case, more than 2000 spectra were recorded over areas of about 300*300 μm and averaged over this area. The Raman spectra obtained were assigned with the aid of literature values to the corresponding phases of Bi, Mo, Co and Fe.

For the spectra shown in FIG. 7 (catalysts Mo₁₂Bi_{1.5}Co₅Fe₃Oₓ of experiments 1 to 3, synthesized hydrothermally at a pH of 6, 7 or 8), FIG. 9 (catalyst Mo₁₂Bi_{1.5}Co₅Fe_{1.8}Oₓ, synthesized by coprecipitation), and FIG. 10, (catalyst Mo₁₂Bi₁₅Co₅Fe₃Oₓ, synthesized hydrothermally at pH=7), 30 mg of catalyst having a particle size of 150 to 300 μm were introduced in each case into a quartz glass capillary of diameter 2 mm. A gas stream of 10 ml per minute having the composition of N₂/O₂/C₃H₆/H₂ in a ratio of 72/18/8/8 was passed through this quartz glass capillary. The quartz glass capillary was heated with an Oxford gas blower to a temperature of up to 445° C. The laser beam was focused onto this capillary with the aid of a video fibre-optic from Renishaw GmbH connected to the Raman spectrometer. The spectra in FIG. 7 were each recorded at 80° C. before (dark line) and after the catalysed reaction (light line). The spectra in FIGS. 9 and 10 were recorded during the catalysed reaction, with the following temperature profile defined by means of the gas blower: 180° C. (0 min)→4 K min⁻¹→320° C. (20 min)→4 K min⁻¹→345° C. (46 min)→4 K min⁻¹→370° C. (46 min)→4 K min⁻¹→395° C. (46 min)→4 K min⁻¹→420° C. (23 min)→4 K min⁻¹→445° C. (23 min)→−10 K min⁻¹→180° C. (18 min)→−10 K min⁻¹→60° C.

### Example 6

### Testing of the Catalysts According to the Invention

The catalysts according to the invention of experiments 1 to 3 (HS-Bi_{1.5}Mo₁₂Co₅Fe₃Oₓ-pH6, HS-Bi_{1.5}Mo₁₂Co₅Fe₃Oₓ-pH7, and HS-Bi_{1.5}Mo₁₂Co₅Fe₃Oₓ-pH8), the catalyst Mo₁₂Bi_{1.5}Co₅Fe₃Oₓ prepared by means of coprecipitation according to WO 2007/042369 A and the binary catalyst prepared Bi₁Mo₁Oₓ by means of hydrothermal synthesis (HS-Bi₁Mo₁Oₓ-pH6) were tested in the partial gas phase oxidation of propene. For each test, a reactor having an internal diameter of 6 mm was charged with 800 mg in each case of the catalyst to be tested of a sieve fraction from 300 to 450 μm, such that its interior was filled essentially completely by the catalyst. The resulting fixed catalyst bed had a height of 2.7 cm. There was one thermocouple each at the upper and lower ends of the fixed catalyst bed, which was in contact with the fixed catalyst bed. Via an external heat supply, the temperature of the fixed catalyst bed was adjusted to a value of about 380° C. Each reactor charged with a specific catalyst was supplied in three experiments with a reactant gas mixture of the composition N₂/O₂/C₃H₆/H₂ in a ratio of 70/14/8/8 with a flow rate of 100, 150 and 200 ml (STP) per minute; based on the mass of the respective catalyst used, the modified residence time (as a quotient of mass of catalyst used divided by the flow rate) for the different flow rates was thus 0.48 g s ml⁻¹, 0.32 g s ml⁻¹ and 0.24 g s ml⁻¹ respectively. During the test, the temperature of the fixed catalyst bed was kept essentially constant at 380° C. by, in the event of temperature fluctuations in the fixed catalyst bed, correspondingly adjusting the temperature of the external heat source. The reaction mixture leaving the reactor was passed via a heated conduit to an Agilent 7890B gas chromatograph.

The gas chromatograph was equipped with two sample loops for permanent gases, especially nitrogen, oxygen, carbon monoxide and carbon dioxide, and for hydrocarbons, especially propene, acrolein and acrylic acid. The first sample loop consisted of two series-connected Agilent Hayesep Q separation columns and a final micro-packed column of the Agilent Molsieve 5A type. The first Hayesep Q separation column served to remove permanent gases from the hydrocarbons; the second Hayesep Q separation column served to separate the permanent gases: carbon dioxide was the first gas eluted from the second Hayesep Q separation column, and then the other permanent gases were eluted together from the second Hayesep Q separation column and passed through a valve to a final separation column of the Molsieve 5A type which served for separation of the permanent gases N₂, O₂, CO, CO₂ and the permanent gases eluted from the Molsieve 5A separation column were detected with a thermal conductivity detector. The second sample loop consisted of an Agilent HP-FFAP capillary column. The compounds eluted from this separation column, propene, acrolein and acrylic acid, were detected with a flame ionization detector. The gas chromatograph was calibrated with gas mixtures of known concentration of N₂, CO, CO₂, propene and propane and a gas mixture of N₂ and O₂, and with standard solutions of propene, acrolein and acrylic acid in methanol. This calibration was followed by the separation and determination of the individual components of the reaction mixtures in the individual catalyst tests. The amounts of propene used and the amounts of propene, acrolein, acrylic acid and carbon oxides CO and CO₂ detected were used to ascertain the propene conversion and the selectivities for the formation of acrolein, acrylic acid and carbon oxides CO and CO₂. These values are compiled in Tables 2 to 4 below, wherein HS indicates obtained by hydrothermal synthesis and CP indicates obtained by coprecipitation.

TABLE 2
Overview of the catalytic properties of the catalysts tested at w/F = 0.48 g s ml⁻¹.

| | | | | | | T(cat.) | T(cat.) | |
|---|---|---|---|---|---|---|---|---|
| | Propylene | Acrolein | CO + CO₂ | Acrylic acid | Oven | at | at | |
| Catalyst | conversion/% | selectivity/% | selectivity/% | selectivity/% | temperature/° C. | inlet/° C. | outlet/° C. | ΔT(cat)/K |
| CP—Mo₁₂Bi_{1.5}Co₅Fe₃Oₓ | 47.0 | 63.5 | 27.7 | 8.3 | 373 | 395 | 366 | 29 |
| HS—Bi_{1.5}Mo₁₂Co₅Fe₃Oₓ-pH 6 | 29.0 | 50.5 | 44.6 | 4.4 | 348 | 363 | 393 | 30 |
| (experiment 1) | | | | | | | | |
| HS—Bi_{1.5}Mo₁₂Co₅Fe₃Oₓ-pH 7 | 63.4 | 75.9 | 20.9 | 2.8 | 354 | 376 | 384 | 8 |
| (experiment 2) | | | | | | | | |
| HS—Bi_{1.5}Mo₁₂Co₅Fe₃Oₓ-pH 8 | 52.6 | 59.7 | 25.7 | 14.2 | 370 | 378 | 383 | 5 |
| (experiment 3) | | | | | | | | |
| HS—Bi₁Mo₁Oₓ-pH 6 | 18.1 | 79.7 | 19.7 | 0.0 | 380 | 377 | 383 | 6 |

TABLE 3
Overview of the catalytic properties of the catalysts tested at w/F = 0.32 g s ml⁻¹.

| | | | | | | T(cat.) | T(cat.) | |
|---|---|---|---|---|---|---|---|---|
| | Propylene | Acrolein | CO + CO₂ | Acrylic acid | Oven | at | at | |
| Catalyst | conversion/% | selectivity/% | selectivity/% | selectivity/% | temperature/° C. | inlet/° C. | outlet/° C. | ΔT(cat)/K |
| CP—Mo₁₂Bi_{1.5}Co₅Fe₃Oₓ | 38.0 | 65.7 | 20.2 | 13.8 | 371 | 390 | 370 | 20 |
| HS—Bi_{1.5}Mo₁₂Co₅Fe₃Oₓ-pH 6 | 19.6 | 63.3 | 36.3 | 0.0 | 350 | 361 | 397 | 36 |
| (experiment 1) | | | | | | | | |
| HS—Bi_{1.5}Mo₁₂Co₅Fe₃Oₓ-pH 7 | 56.4 | 77.2 | 18.0 | 4.6 | 353 | 378 | 383 | 5 |
| (experiment 2) | | | | | | | | |
| HS—Bi_{1.5}Mo₁₂Co₅Fe₃Oₓ-pH 8 | 41.3 | 69.1 | 21.5 | 9.2 | 369 | 373 | 387 | 14 |
| (experiment 3) | | | | | | | | |
| HS—Bi₁Mo₁Oₓ-pH 6 | 12.5 | 94.0 | 5.6 | 0.0 | 381 | 377 | 384 | 7 |

TABLE 4
Overview of the catalytic properties of the catalysts tested at w/F = 0.24 g s ml⁻¹.

| | | | | | | T(cat.) | T(cat.) | |
|---|---|---|---|---|---|---|---|---|
| | Propylene | Acrolein | CO + CO₂ | Acrylic acid | Oven | at | at | |
| Catalyst | conversion/% | selectivity/% | selectivity/% | selectivity/% | temperature/° C. | inlet/° C. | outlet/° C. | ΔT(cat)/K |
| CP—Mo₁₂Bi_{1.5}Co₅Fe₃Oₓ | 32.0 | 70.5 | 19.0 | 10.1 | 370 | 388 | 368 | 20 |
| HS—Bi_{1.5}Mo₁₂Co₅Fe₃Oₓ-pH 6 | 16.2 | 62.8 | 33.0 | 3.4 | 353 | 360 | 400 | 40 |
| (experiment 1) | | | | | | | | |
| HS—Bi_{1.5}Mo₁₂Co₅Fe₃Oₓ-pH 7 | 47.5 | 79.3 | 14.9 | 5.6 | 350 | 375 | 385 | 10 |
| (experiment 2) | | | | | | | | |
| HS—Bi_{1.5}Mo₁₂Co₅Fe₃Oₓ-pH 8 | 33.8 | 72.5 | 19.0 | 8.3 | 369 | 370 | 390 | 20 |
| (experiment 3) | | | | | | | | |
| HS—Bi₁Mo₁Oₓ-pH 6 | 10.9 | 96.0 | 2.7 | 0.0 | 381 | 377 | 384 | 7 |

1. A process for preparing a multielement oxide catalyst, the process comprising:
providing a mixture of an aqueous solution and/or an aqueous suspension of precursor compounds of elements present in the multielement oxide catalyst in an amount to achieve stoichiometry thereof,
setting a pH of the mixture obtained from the providing to a value between 5.5 and 8.5,
reacting the mixture comprising the precursor compounds obtained from the setting under solvothermal reaction conditions in an autoclave at a temperature of from 100° C. to 600° C. to form the multielement oxide catalyst, and
separating the multielement oxide catalyst from the aqueous solution and/or suspension,
wherein the multielement oxide catalyst is of formula (I):
MoₐBi_{b}Co_{c}Fe_{d}Ni_{g}X_{f}X′_{g}X″ₕX′″ᵢX″″ⱼOₓ (I),
wherein
X is W or P,
X′ is Li, K, Na, Rb, Cs, Mg, Ca, Ba or Sr,
X″ is Ce, Mn, Cr or V,
X″′ is Nb, Se, Te, Sm, Gd, La, Y, Pd, Pt, Ru, Ag or Au,
X″″ is Si, Al, Ti or Zr,
a is 12,
b is 1 to 4,
c is 4 to 10,
d is 1 to 4,
e is 0 to 4,
f is 0 to 5,
g is 0 to 2,
h is 0 to 5,
i is 0 to 2,
j is 0 to 800, and
x is a number which is determined by a valency and frequency of the elements other than oxygen.

2. The process according to claim 1, wherein the precursor compounds in step a) are salts.

3. The process according to claim 1, wherein the setting is setting a pH of of the mixture to a value between 6.5 and 7.5.

4. The process according to claim 1, wherein the reacting is reacting at a temperature of from 100° C. to 400° C.

5. The process according to claim 1, wherein the reacting is conducted for a period of from 5.5 to 48.5 hours.

6. The process according to claim 1, further comprising:
washing the multielement oxide catalyst obtained from the separating at least once, and
drying and/or calcining the multielement oxide catalyst.

7. The process according to claim 1, further comprising:
applying the multielement oxide catalyst obtained from the separating, the washing, or the drying and/or calcining to a support to obtain a supported catalyst,
or
forming the multielement oxide catalyst obtained from the separating, the washing, or the drying and/or calcining to obtain an unsupported catalyst,
and
additionally drying and/or calcining the multielement oxide catalyst obtained from the applying or the forming.

8. A multielement oxide catalyst of formula:
MoₐBi_{b}Co_{c}Fe_{d}NiₑX_{f}X′_{g}X″ₕX″′ᵢX″″ᵢOₓ,
wherein
X is W or P,
X′ is Li, K, Na, Rb, Cs, Mg, Ca, Ba or Sr,
X″ is Ce, Mn, Cr or V,
X′″ is Nb, Se, Te, Sm, Gd, La, Y, Pd, Pt, Ru, Ag or Au,
X″″ is Si, Al, Ti or Zr, and
a is 12,
b is 1 to 4,
c is 4 to 10,
d is 1 to 4,
e is 0 to 4,
f is 0 to 5,
g is 0 to 2,
h is 0 to 5,
i is 0 to 2,
j is 0 to 800, and
x is a number which is determined by a valency and frequency of the elements other than oxygen, and
wherein the multielement oxide catalyst comprises areas, in which molybdenum, bismuth and iron are simultaneously present, and the areas have a diameter of from 10 nm to 25 μm.

9. A multielement oxide catalyst obtained by the process according to claim 1, wherein a pH of between 6.5 and 7.5is set in the setting.

10. The multielement oxide catalyst according to claim 8, wherein e to i are each 0.

11. The multielement oxide catalyst according to claim 8, wherein a is 12, b is 1 to 2, c is 5 to 8 and d is 2 to 3.

12. The multielement oxide catalyst according to claim 8, wherein the multielement oxide catalyst does not contain a MoO₃-phase.

13. The multielement oxide catalyst according to claim 8, wherein the multielement oxide catalyst has an alpha-bismuth molybdate phase and a beta-cobalt molybdate phase.

14. A process, comprising:
performing a partial gas phase oxidation and/or an ammoxidation of olefins or tert-butanol in presence of the multielement oxide catalyst according to claim 8.

15. The process according to claim 14, wherein the olefin is propene and/or isobutene.
